(19) United States
(12) Patent Application Publication
(10) Pub. No.: US 2002/0034483 A1
(43) Pub. Date: Mar. 21, 2002

(54) Hair treatment compositions

(75) Inventors: Andrew Richard Avery, Bebington (GB); Andrew Anthony Howard Barnes, Bebington (GB); Andrew Malcolm Murray, Bebington (GB); Malika Punyagupta, Suanluang (TH)

Correspondence Address: UNILEVER, PATENT DEPARTMENT, 45 RIVER ROAD, EDGEWATER, NJ, 07020, US

(73) Assignee: Unilever Home & Personal Care USA,

(21) Appl. No.: 09/861,485

(22) Filed: May 18, 2001

(30) Foreign Application Priority Data
May 18, 2000 (GB) 0012061.8

(51) Int. Cl. A61K 7/06; A61K 7/00

(52) U.S. Cl. 424/70.1; 424/400; 424/401

(57) ABSTRACT

Hair treatment compositions comprising a surfactant, a dispersed insoluble benefit agent, and PTFE microparticles. The use of PTFE microparticles as a deposition aid for a dispersed insoluble benefit agent in an aqueous hair treatment composition is also described.

### FIELD OF THE INVENTION

The invention relates to hair treatment compositions for increased deposition of a benefit agent dispersed in the composition onto the hair, which compositions comprise PTFE microparticles.

### BACKGROUND AND PRIOR ART

Many personal care compositions contain benefit agents which need to be delivered to and deposited onto a target surface, i.e., skin or hair. The composition must leave the benefit agent on the target surface after the product is washed and rinsed off the surface.

Various attempts have been made to improve the efficiency of use of expensive benefit agents such as silicones. This would provide better conditioning and the option of reducing the level of expensive benefit agent in the composition, with consequent cost saving.

Deposition polymers with a cationic charge have been proposed to enhance the amount of benefit agent deposited from shampoo. For example cationic guar gum has been described for the enhancement of the deposition of antidandruff particles in U.S. Pat. No. 5,037,818 and for the enhanced deposition of insoluble non-volatile silicone in U.S. Pat. No. 5,085,857. Cationic polymers have been proposed to enhance the deposition of sunscreen materials from a shampoo composition. In EP 386 898 a cationic polygalactomannan gum derivative is used. The use of cationic polymers in shower gels to enhance deposition of silicone oil is also known from EP-A-457 688 (L'Oreal).

Unfortunately, a great number of personal care compositions contain anionic surfactants. Anionic surfactants interfere with deposition by adsorbing on all surfaces as well as forming complexes/precipitates with cationic deposition aids. Even if deposition occurs, the formulations may exhibit poor stability due to flocculation and precipitation.

The higher the concentration of anionic surfactant, the harder it is to attain deposition of benefit agents. Thus, it is desirable to improve the deposition of benefit agents onto a target surface such as skin or hair in the presence of an anionic surfactant.

The present inventors have found that the efficiency of deposition of a dispersed insoluble benefit agent from a shampoo or hair conditioner can be significantly enhanced by the inclusion in the composition of PTFE microparticles.

PTFE per se has previously been described in personal care compositions, as follows:

U.S. Pat. No. 3,568,685 describes a composition for straightening hair, consisting of a water-repellent agent, a hardening and adhesive agent, an emollient and a slipping agent which may be any one of a number of fluoro resins, such as vinylidene fluoride resin or PTFE.

U.S. Pat. No. 3,911,106 describes a method of conditioning hair and scalp by rubbing in PTFE of specified molecular weight. The PTFE may be used by itself, in aqueous composition or diluted with a suitable diluent such as fatty alcohol or mineral oil.

PTFE microparticles have been described in U.S. Pat. No. 4,047,537 describes the use of a colloidal aqueous dispersion of PTFE particles as a hair control agent for promoting hair body, fullness and set retention. The PTFE particles may be used directly on the hair or formulated with an oil-free or lower primary alcohol-free liquid which is preferably a water soluble shampoo.

None of the above documents disclose or suggest the utility of PTFE microparticles as a deposition aid for dispersed insoluble benefit agents in aqueous hair treatment compositions such as shampoos or hair conditioners.

### SUMMARY OF THE INVENTION

The present invention provides, in a first aspect, an aqueous hair treatment composition comprising:

(i) at least one surfactant;

(ii) at least one dispersed insoluble benefit agent, and

(iii) PTFE microparticles.

In a second aspect, the invention provides the use of PTFE microparticles as a deposition aid for a dispersed insoluble benefit agent in an aqueous hair treatment composition.

### DETAILED DESCRIPTION AND PREFERRED EMBODIMENTS

### PTFE MICROPARTICLES

By “PTFE microparticles” is meant PTFE containing particles which may range in size from 0.01 up to 10 microns, preferably from 0.05 up to 10 microns.

Preferably the PTFE microparticles are present in the composition of the invention in the form of a colloidal dispersion thereof. Typically the primary particle size of the PTFE microparticles in such a colloidal dispersion will range from 0.05 up to 0.5 microns, with an average diameter of preferably about 0.2 microns.

The microparticles may be composed entirely of PTFE polymer, or may consist of a composite of PTFE polymer and one or more further polymers such as polyethylene (PE).

By “PTFE polymer” is meant a polymer consisting of:

(a) 95 to 100%, preferably substantially 100%, of units derived from tetrafluoroethylene, and

(b) optionally, up to 5%, preferably not more than 2%, of units derived from a copolymerizable monomer, e.g. hexafluoropropylene, perfluorinated vinyl ether, hexafluoroisobutylene, vinylidene fluoride, or an olefin.

A preferred source of PTFE microparticles for inclusion in compositions of the invention is a pre-formed colloidal dispersion of PTFE microparticles.

Such dispersions are commercially available, for example those sold by Ausimont under the trade name ALGOFLON, such as ALGOFLON D60G, and aqueous dispersions of PTFE sold by Du Pont such as Teflon®30-N.

Also suitable is the series of materials sold by Micropowders, Inc. under the tradename MICROSILK, such as MICROSILK 419.

The PTFE microparticles are typically present in compositions of the invention at a level of from 0.05% to 10%, preferably from 0.1% to 4%, more preferably from about 1% to 2%, by total weight of PTFE microparticles based on total weight of the composition.

### BENEFIT AGENT

Compositions according to the present invention comprise a dispersed insoluble benefit agent.

This component will be dispersed in the composition in the form of droplets, which form a separate, discontinuous phase from the aqueous, continuous phase of the composition.

By “insoluble” is meant that the material is not soluble in water (distilled or equivalent) at a concentration of 0.1%, at 250° C.

The benefit agent may suitably be selected from one or more of the following classes of material: (a) conditioning agents, (b) solid active agents, and mixtures thereof.

(a) Conditioning Agents

As used herein, the term “conditioning agent” includes any material which is used to give a particular conditioning benefit to hair and/or skin. For example, in compositions for use in washing hair, such as shampoos and conditioners, suitable materials are those which deliver one or more benefits relating to shine, softness, combability, wet-handling, anti-static properties, protection against damage, body, volume, stylability and manageability.

The conditioning agent may suitably be selected from one or more of the following classes of material:

(i) silicones, (ii) high molecular weight hydrocarbon materials, (iii) oily or fatty materials, and mixtures thereof.

(i) Silicones

Silicone is a particularly preferred conditioning agent for hair treatment compositions according to the invention. In particular, hair shampoos and conditioners of the invention will preferably comprise emulsified particles of silicone, for enhancing conditioning performance. The silicone is insoluble in the aqueous matrix of the composition and so is present in an emulsified form, with the silicone present as dispersed particles.

Suitable silicones include polydiorganosiloxanes, in particular polydimethylsiloxanes which have the CTFA designation dimethicone. Also suitable for use in compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188. These materials can impart body, volume and stylability to hair, as well as good wet and dry conditioning.

The viscosity of the emulsified silicone itself (not the emulsion or the final hair conditioning composition) is typically at least 10,000 cst. In general we have found that conditioning performance increases with increased viscosity. Accordingly, the viscosity of the silicone itself is preferably at least 60,000 cst, most preferably at least 500,000 cst, ideally at least 1,000,000 cst. Preferably the viscosity does not exceed 10⁹ cst for ease of formulation.

Emulsified silicones for use in hair shampoos and conditioners of the invention will typically have an average silicone particle size in the composition of less than 30, preferably less than 20, more preferably less than 10 microns. We have found that reducing the particle size generally improves conditioning performance. Most preferably the average silicone particle size of the emulsified silicone in the composition is less than 2 microns, ideally it ranges from 0.01 to 1 micron. Silicone emulsions having an average silicone particle size of ≦0.15 microns are generally termed microemulsions.

Particle size may be measured by means of a laser light scattering technique, using a 2600D Particle Sizer from Malvern Instruments.

Suitable silicone emulsions for use in the invention are also commercially available in a pre-emulsified form.

Examples of suitable pre-formed emulsions include emulsions DC2-1766, DC2-1784, and microemulsions DC2-1865 and DC2-1870, all available from Dow Corning. These are all emulsions/microemulsions of dimethiconol. Cross-linked (C) WO COM silicone gums are also available in a pre-emulsified form, which is advantageous for ease of formulation. A preferred example is the material available from Dow Corning as DC X2-1787, which is an emulsion of cross-linked dimethiconol gum. A further preferred example is the material available from Dow Corning as DC X2-1391, which is a microemulsion of cross-linked dimethiconol gum.

A further preferred class of silicones for inclusion in shampoos and conditioners of the invention are amino functional silicones. By “amino functional silicone” is meant a silicone containing at least one primary, secondary or tertiary amine group, or a quaternary ammonium group.

Examples of suitable amino functional silicones include:

(i) polysiloxanes having the CTFA designation “amodimethicone”, and the general formula:

HO—[Si(CH₃)₂—O—]ₓ—[Si(OH)(CH₂CH₂CH₂—NH—CH₂CH₂NH₂)—O—]_{y}—H

in which x and y are numbers depending on the molecular weight of the polymer, generally such that the molecular weight is between about 5,000 and 500,000.

(ii) polysiloxanes having the general formula:

R′ₐG₃—ₐ—Si(OSiG₂)n—(OSiG_{b}R′_{2-b})ₘ—O—SiG₃₋ₐ—R′a

in which:

G is selected from H, phenyl, OH or C₁₋₈ alkyl, e.g. methyl;

a is 0 or an integer from 1 to 3, preferably 0;

b is 0 or 1, preferably 1;

m and n are numbers such that (m+n) can range from 1 to 2000, preferably from 50 to 150;

m is a number from 1 to 2000, preferably from 1 to 10;

n is a number from 0 to 1999, preferably from 49 to 149, and

R′ is a monovalent radical of formula —C_{q}H_{2q}L in which q is a number from 2 to 8 and L is an aminofuctional group selected from the following:

—NR″—CH₂—CH₂—N(R″)2

—N(R″)2

—N⁺(R″)₃A⁻

—N⁺H(R″)₂A⁻

—N⁺H₂(R″) A⁻

—N(R″)—CH₂—CH₂—N⁺H₂(R″) A⁻

in which R″ is selected from H, phenyl, benzyl, or a saturated monovalent hydrocarbon radical, e.g. C₁₋₂₀ alkyl, and

A is a halide ion, e.g. chloride or bromide.

Suitable amino functional silicones corresponding to the above formula include those polysiloxanes termed “trimethylsilylamodimethicone” as depicted below, and which are sufficiently water insoluble so as to be useful in compositions of the invention:

Si(CH₃)₃—O—[Si(CH₃)₂—O—]x—[Si (CH₃) (R—NH—CH₂CH₂ NH₂)—O—]_{y}—Si (CH₃)₃

wherein x+y is a number from about 50 to about 500, and wherein R is an alkylene group having from 2 to 5 carbon atoms. Preferably, the number x+y is in the range of from about 100 to about 300.

(iii) quaternary silicone polymers having the general formula:

{(R¹) (R²) (R³) N⁺CH₂CH(OH)CH₂O(CH₂)₃[Si (R⁴) (R⁵)—O—]n—Si(R⁶)(R⁷)—(CH₂)₃—O—CH₂CH(OH)CH₂N³⁰ (R⁸) (R⁹) (R¹⁰)} (X⁻)₂

wherein R¹ and R¹⁰ may be the same or different and may be independently selected from H, saturated or unsaturated long or short chain alk(en)yl, branched chain alk(en)yl and C₅-C₈ cyclic ring systems;

R² thru R⁹ may be the same or different and may be independently selected from H, straight or branched chain lower alk(en)yl, and C₅-C₈ cyclic ring systems;

n is a number within the range of about 60 to about 120, preferably about 80, and

X″ is preferably acetate, but may instead be for example halide, organic carboxylate, organic sulphonate or the like.

Suitable quaternary silicone polymers of this class are described in EP-A-0 530 974.

Amino functional silicones suitable for use in shampoos and conditioners of the invention will typically have a mole % amine functionality in the range of from about 0.1 to about 8.0 mole preferably from about 0.1 to about 5.0 mole most preferably from about 0.1 to about 2.0 mole In general the amine concentration should not exceed about 8.0 mole % since we have found that too high an amine concentration can be detrimental to total silicone deposition and therefore conditioning performance.

The viscosity of the amino functional silicone is not particularly critical and can suitably range from about 100 to about 500,000 cst.

Specific examples of amino functional silicones suitable for use in the invention are the aminosilicone oils DC2-8220, DC2-8166, DC2-8466, and DC2-8950-114 (all ex Dow Corning), and GE 1149-75, (ex General Electric Silicones).

Also suitable are emulsions of amino functional silicone oils with non ionic and/or cationic surfactant.

Suitably such pre-formed emulsions will have an average amino functional silicone particle size in the shampoo composition of less than 30, preferably less than 20, more preferably less than 10 microns. Again, we have found that reducing the particle size generally improves conditioning performance. Most preferably the average amino functional silicone particle size in the composition is less than 2 microns, ideally it ranges from 0.01 to 1 micron. Silicone emulsions having an average silicone particle size of ≦0.15 microns are generally termed microemulsions.

Pre-formed emulsions of amino functional silicone are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2-8154 (all ex Dow Corning).

An example of a quaternary silicone polymer useful in the present invention is the material K3474, ex Goldschmidt.

For shampoo compositions according to the invention intended for the treatment of “mixed” hair (i.e. greasy roots and dry ends), it is particularly preferred to use a combination of amino functional and non-amino functional silicone in compositions of the invention, especially when these are in the form of shampoo compositions. In such a case, the weight ratio of amino functional silicone to non-amino functional silicone will typically range from 1:2 to 1:20, preferably 1:3 to 1:20, more preferably 1:3 to 1:8.

The total amount of silicone incorporated into compositions of the invention depends on the level of conditioning desired and the material used. A preferred amount is from 0.01 to about 10% by weight of the total composition although these limits are not absolute. The lower limit is determined by the minimum level to achieve conditioning and the upper limit by the maximum level to avoid making the hair and/or skin unacceptably greasy.

We have found that a total amount of silicone of from 0.3 to 5%, preferably 0.5 to 3%, by weight of the total composition is a suitable level.

(ii) High Molecular Weight Hydrocarbon Materials

By “high molecular weight” is meant that the weight average molecular weight of the emulsified hydrocarbon material is at least 20,000. Suitably it ranges from 20,000 to 1,000,000, preferably 20,000 to 500,000, most preferably 40,000 to 200,000. These materials are especially effective for imparting improved fullness, body and volume to hair.

A preferred class of high molecular weight hydrocarbon materials are per-alk(en)yl hydrocarbon resins. These term “resin” is intended to encompass those materials which are solid or semi-solid at room temperature, as well as those which are liquids with high or moderate viscosities. The term does not cover oils or other low viscosity materials.

EP 567 326 and EP 498 119 describe suitable peralk(en)yl hydrocarbon resins for imparting stylability and enhanced body to hair. Preferred per-alk(en)yl hydrocarbon materials are polymers of butene, isoprene, terpene and styrene, and copolymers of any combination of these monomers, such as butyl rubber (poly isobutylene-co-isoprene), natural rubber (cis-1,4-polyisoprene) and hydrocarbon resins such as mentioned in the Encyclopedia of Chemical technology by Kirk and Othmer (3rd edition vol.8, pp 852-869), for example aliphatic and aromatic resins and terpene resins.

Especially preferred are polyisobutylene materials of the formula:

H₃C —[C(CH₃)₂—CH₂−]ₘ—R

wherein m is 1-5000, preferably 2-2500, and R is:

—CH(CH₃)₂ or —C(CH₃)=CH₂

These materials are available from Presperse, Inc. under the PERMETHYL trade name, from Exxon Chemical under the VISTANEX trade name, and from BASF under the OPANOL trade name. Preferred examples include VISTANEX LM-MH and OPANOL B 15.

Suitable methods of making emulsions of particles of high molecular weight hydrocarbon materials such as polyisobutylene resins are described in EP 567 326 and EP 498 119. The process of EP 567 326 is preferred since it is a direct emulsification process with water and a suitable surfactant emulsifier which avoids the need to use a solvent or carrier which is capable of dissolving or dispersing the high molecular weight hydrocarbon material. Such solvents or carriers (e.g. low molecular weight hydrocarbons) can present safety hazards during processing and can destabilise the final formulations into which they are incorporated.

Emulsified high molecular weight hydrocarbon materials for use in hair treatment compositions of the invention generally have an average particle size in the composition of from about 0.01 to about 100 microns, more typically from about 0.1 to about 10 microns, thought this is not particularly critical. Particle size may be measured by means of a laser light scattering technique, using a 2600D Particle Sizer from Malvern Instruments.

Suitable high molecular weight hydrocarbon emulsions for use in the invention are commercially available in a pre-emulsified form. This is particularly preferred since the pre-formed emulsion can be incorporated into the hair treatment composition by simple mixing.

An example of a suitable pre-formed emulsion is the material PIB 96/003 available from Basildon Chemical. This is an aqueous emulsion of the polyisobutylene material OPANOL B 15 (ex BASF) with anionic and nonionic surfactant emulsifier.

The high molecular weight hydrocarbon material may be present in compositions of the invention as a single material or as a mixture of different high molecular weight hydrocarbon materials, e.g. of different molecular weights.

The amount of high molecular weight hydrocarbon material incorporated into the compositions of the invention depends on the level of fullness, body and volume enhancement desired and the specific material used. A preferred amount is from about 0.01 to about 2% by weight of the total composition although these limits are not absolute. The lower limit is determined by the minimum level to achieve the fullness, body and volume enhancing effect and the upper limit by the maximum level to avoid making the hair unacceptably stiff. We have found that an amount of high molecular weight hydrocarbon material of from 0.2 to 0.5% by weight of the total composition is a particularly suitable level.

(iii) Oily or Fatty Materials

Oily or fatty materials are preferred conditioning agents in hair treatment compositions of the invention for adding shine to the hair and also enhancing dry combing and dry hair feel.

Suitable oily or fatty materials will generally have a viscosity at ambient temperature of about 3 million cst or less, preferably about 2 million cst or less, more preferably about 1.5 million cst or less. However, fatty materials which are solid at ambient temperature may also be suitable.

Suitable oily or fatty materials are selected from hydrocarbon oils, fatty esters and mixtures thereof.

Hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will preferably contain from about 12 to about 19 carbon atoms. Branched chain hydrocarbon oils can and typically may contain higher numbers of carbon atoms. Also suitable are polymeric hydrocarbons of alkenyl monomers, such as C₂ -C₆ alkenyl monomers. These polymers can be straight or branched chain polymers. The straight chain polymers will typically be relatively short in length, having a total number of carbon atoms as described above for straight chain hydrocarbons in general. The branched chain polymers can have substantially higher chain length. The number average molecular weight of such materials can vary widely, but will typically be up to about 500, preferably from about 200 to about 400, more preferably from about 300 to about 350.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used. Exemplary branched-chain isomers are highly branched saturated or unsaturated alkanes, such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, sold by Permethyl Corporation. A further example of a hydrocarbon polymer is polybutene, such as the copolymer of isobutylene and butene. A commercially available material of this type is L-14 polybutene from Amoco Chemical Co. (Chicago, Ill., U.S.A.).

Particularly preferred hydrocarbon oils are the various grades of mineral oils. Mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons ranging from C₁₆H₃₄ to C₂₁H₄₄. Suitable commercially available materials of this type include Sirius M85 and Sirius M125, all available from Silkolene.

Suitable fatty esters are characterised by having at least 10 carbon atoms, and include esters with hydrocarbyl chains derived from fatty acids or alcohols, e.g., monocarboxylic acid esters, polyhydric alcohol esters, and di- and tricarboxylic acid esters. The hydrocarbyl radicals of the fatty esters hereof can also include or have covalently bonded thereto other compatible functionalities, such as amides and alkoxy moieties, such as ethoxy or ether linkages.

Monocarboxylic acid esters include esters of alcohols and/or acids of the formula R′COOR in which R′ and R independently denote alkyl or alkenyl radicals and the sum of carbon atoms in R′ and R is at least 10, preferably at least 20.

Specific examples include, for example, alkyl and alkenyl esters of fatty acids having aliphatic chains with from about 10 to about 22 carbon atoms, and alkyl and/or alkenyl fatty alcohol carboxylic acid esters having an alkyl and/or alkenyl alcohol-derived aliphatic chain with about 10 to about 22 carbon atoms, and mixtures thereof.

The monocarboxylic acid ester need not necessarily contain at least one chain with at least 10 carbon atoms, so long as the total number of aliphatic chain carbon atoms is at least 10. Examples include isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Di- and trialkyl and alkenyl esters of carboxylic acids can also be used. These include, for example, esters of C₄ -C₈ dicarboxylic acids such as C₁-C₂₂ esters (preferably C₁ -C₆) of succinic acid, glutaric acid, adipic acid, hexanoic acid, heptanoic acid, and octanoic acid. Examples include diisopropyl adipate, diisohexyl adipate, and diisopropyl sebacate. Other specific examples include isocetyl stearoyl stearate, and tristearyl citrate.

Polyhydric alcohol esters include alkylene glycol esters, for example ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol monostearate, ethoxylated propylene glycol monostearate, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, polyoxyethylene sorbitan fatty acid esters and mono-, di-and triglycerides.

Particularly preferred fatty esters are mono-, di- and triglycerides, more specifically the mono-, di-, and tri-esters of glycerol and long chain carboxylic acids such as C₁-C₂₂ carboxylic acids. A variety of these types of materials can be obtained from vegetable and animal fats and oils, such as coconut oil, castor oil, safflower oil, cottonseed oil, corn oil, olive oil, cod liver oil, almond oil, avocado oil, palm oil, sesame oil, lanolin and soybean oil. Synthetic oils include triolein and tristearin glyceryl dilaurate. Specific examples of preferred materials include cocoa butter and palm stearin.

The oily or fatty material is typically present at a level of from 0.05% to 10%, preferably from 0.2% to 5%, more preferably from about 0.5% to 3%, by total weight of oily or fatty material based on total weight of the composition.

(b) Solid Active Agents

Examples of typical solid active agents include antimicrobial agents, such as the heavy metal salts of pyridinethione, especially zinc pyridinethione. These substances typically have an average particle diameter of from about 0.2 to about 50 microns, preferably from about 0.4 to about 10 microns.

Where the solid active agent is an antimicrobial agent, such as zinc pyridinethione, this may be suitably be employed in the composition in an amount of from 0.001% to about 1% by weight of the total composition.

Other suitable solid active agents include pigment particles, such as solid dyes or colorants suitable for application to hair, and metal colloids.

### PRODUCT FORM

Hair treatment compositions according to the invention may suitably take the form of shampoos, conditioners, sprays, mousses or lotions. Preferred hair treatment composition forms are shampoos and conditioners.

Shampoo Compositions

A particularly preferred form of composition in accordance with the invention is a shampoo composition.

Anionic Cleansing Surfactant

Shampoo compositions according to the invention will typically comprise one or more anionic cleansing surfactants which are cosmetically acceptable and suitable for topical application to the hair.

Examples of suitable anionic cleansing surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic cleansing surfactants for use in shampoo compositions of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, sodium lauryl ether sulphate(n)EO, (where n ranges from 1 to 3), ammonium lauryl sulphate and ammonium lauryl ether sulphate(n)EO, (where n ranges from 1 to 3).

Mixtures of any of the foregoing anionic cleansing surfactants may also be suitable.

The total amount of anionic cleansing surfactant in shampoo compositions of the invention is generally from 5 to 30%, preferably from 6 to 20%, more preferably from 8% to 16% by weight based on total weight of the shampoo composition.

Co-surfactant

The shampoo composition can optionally include co-surfactants, to help impart aesthetic, physical or cleansing properties to the composition.

A preferred example is an amphoteric or zwitterionic surfactant, which can be included in an amount ranging from 0 to about 8%, preferably from 1 to 4% by weight based on total weight of the shampoo composition.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred example is a nonionic surfactant, which can be included in an amount ranging from 0% to about 8% preferably from 2 to 5% by weight based on total weight of the shampoo composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Other representative nonionic surfactants include mono- or di-alkyl alkanolamides. Examples include coco mono- or di-ethanolamide and coco mono-isopropanolamide.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO—(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

Other sugar-derived nonionic surfactants which can be included in shampoo compositions of the invention include the C₁₀-C₁₈ N-alkyl (C₁-C₆) polyhydroxy fatty acid amides, such as the C₁₂-C₁₈ N-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

Cationic Polymer

Shampoo compositions according to the invention may also optionally include a cationic polymer for further enhancing conditioning performance of the shampoo.

The cationic polymer may be a homopolymer or be formed from two or more types of monomers. The molecular weight of the polymer will generally be between 5,000 and 10,000,000, typically at least 10,000 and preferably in the range 100,000 to about 2,000,000. The polymers will have cationic nitrogen containing groups such as quaternary ammonium or protonated amino groups, or a mixture thereof.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic conditioning polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic conditioning polymers can comprise mixtures of monomer units derived from amine- and/or quaternary ammonium-substituted monomer and/or compatible spacer monomers.

Suitable cationic conditioning polymers include, for example:

copolymers of l-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16.

This material is commercially available from BASF Wyandotte Corp. (Parsippany, N.J., USA) under the LUVIQUAT tradename (e.g. LUVIQUAT FC 370);

copolymers of 1-vinyl-2-pyrrolidine and dimethylaminoethyl methacrylate, referred to in the industry (CTFA) as Polyquaternium-11. This material is available commercially from Gaf Corporation (Wayne, N.J., USA) under the GAFQUAT tradename (e.g., GAFQUAT 755N);

cationic diallyl quaternary ammonium-containing polymers including, for example, dimethyldiallyammonium chloride homopolymer and copolymers of acrylamide and dimethyldiallylammonium chloride, referred to in the industry (CTFA) as Polyquaternium 6 and Polyquaternium 7, respectively;

mineral acid salts of amino-alkyl esters of homo-and co-polymers of unsaturated carboxylic acids having from 3 to 5 carbon atoms, (as described in U.S. Pat. No. 4,009,256);

cationic polyacrylamides(as described in WO95/22311).

Other cationic conditioning polymers that can be used include cationic polysaccharide polymers, such as cationic cellulose derivatives, cationic starch derivatives, and cationic guar gum derivatives.

Cationic polysaccharide polymers suitable for use in compositions of the invention include those of the formula:

A—O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Patent 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (Commercially available from Rhone-Poulenc in their JAGUAR trademark series).

Examples are JAGUAR C13S, which has a low degree of substitution of the cationic groups and high viscosity. JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Preferably the cationic conditioning polymer is selected from cationic cellulose and cationic guar derivatives. Particularly preferred cationic polymers are JAGUAR C13S, JAGUAR C15, JAGUAR C17 and JAGUAR C16 and JAGUAR C162.

The cationic conditioning polymer will generally be present in compositions of the invention at levels of from 0.01 to 5%, preferably from about 0.05 to 1%, more preferably from about 0.08% to about 0.5% by weight.

Conditioners

Alternatively, compositions of the invention may be formulated as a hair conditioner for the treatment of hair (typically after shampooing) and subsequent rinsing.

Cationic Conditioning Surfactant

Hair conditioners according to the invention will typically comprise one or more cationic conditioning surfactants which are cosmetically acceptable and suitable for topical application to the hair.

Cationic conditioning surfactants useful in compositions of the invention contain amino or quaternary ammonium hydrophilic moieties which are positively charged when dissolved in the aqueous composition of the present invention.

Examples of suitable cationic conditioning surfactants are those corresponding to the general formula:

[N(R₁) (R₂) (R₃) (R₄)]⁺(X)⁻

in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

Preferred are cationic conditioning surfactants containing two long alkyl chains and two short alkyl chains or especially those containing one long alkyl chain and three short alkyl chains. The long alkyl chains in such compounds generally have from 12 to 22 carbon atoms, preferably from 16 to 22 carbon atoms, and the corresponding short alkyl chains generally have from 1 to 3 carbon atoms, preferably from 1 to 2 carbon atoms.

The most preferred cationic conditioning surfactants for compositions of the present invention are those selected from cetyl trimethyl ammonium chloride, stearyl trimethyl ammonium chloride, behenyl trimethyl ammonium chloride, and mixtures thereof.

Further examples of suitable cationic conditioning surfactants include the materials having the following CTFA designations: Quaternium-5, Quaternium-8, Quaternium-18, Quaternium-24, Quaternium-26, Quaternium-27, Quaternium-30, Quaternium-31, Quaternium-33, Quaternium-43, Quaternium-52, Quaternium-53, Quaternium-56, Quaternium-60, Quaternium-62, Quaternium-70, Quaternium-72, Quaternium-75, Quaternium-77, Quaternium-78, Quaternium-79, Quaternium-80, Quaternium-81, Quaternium-82, Quaternium-83, Quaternium-84, and mixtures thereof.

Salts of primary, secondary and tertiary fatty amines are also suitable cationic conditioning surfactants. The alkyl groups of such amines preferably have from 12 to 22 carbon atoms, and can be substituted or unsubstituted.

Preferred are acid-neutralised amidoamine compounds, wherein the amidoamine compound has the general formula:

R₅—C (O)—NH—R₆—N(R₇)(R₈)

wherein R₅ is a fatty acid chain containing from 12 to 22 carbon atoms, R₆ is an alkylene group containing from one to four carbon atoms, and R₇ and R₈ are, independently, an alkyl group having from one to four carbon atoms.

Examples of suitable amidoamine compounds of the above general formula include stearamidopropyl dimethylamine, stearamidopropyl diethylamine, stearamidoethyl dimethylamine, stearamidoethyl diethylamine, palmitamidopropyl dimethylamine, behenamidopropyl dimethylamine, myristamidopropyl dimethylamine, oleamidopropyl dimethylamine, ricinoleamidopropyl dimethylamine, and combinations thereof.

The acid used to neutralise the amidoamine compound can be essentially any organic acid or mineral acid of sufficient acid strength to neutralise a free amine nitrogen. Such acids include hydrochloric acid, sulphuric acid, nitric acid, phosphoric acid, lactic acid, citric acid, tartaric acid, acetic acid, gluconic acid, glycolic acid and propionic acid, or combinations thereof. Lactic acid is a preferred neutraliser since it can provide superior composition stability.

Mixtures of any of the foregoing cationic conditioning surfactants may also be suitable.

In compositions of the invention, the level of cationic conditioning surfactant is preferably from 0.01 to 10%, more preferably 0.05 to 5%, most preferably 0.1 to 3% by weight based on total weight of the composition.

Fatty Alcohol Material

Conditioners according to the invention may advantageously incorporate a fatty alcohol material.

By “fatty alcohol material” is meant a fatty alcohol, an alkoxylated fatty alcohol, or a mixture thereof.

Representative fatty alcohols comprise from 8 to 22 carbon atoms more preferably 16 to 20. Examples of suitable fatty alcohols include cetyl alcohol, stearyl alcohol and mixtures thereof. The use of these materials is also advantageous in that they contribute to the overall conditioning properties of compositions of the invention.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (24) cetyl ether, and mixtures thereof.

The level of fatty alcohol material in conditioners of the invention is suitably from 0.01 to 15%, preferably from 0.1 to 10% by weight based on total weight of the composition.

The weight ratio of cationic conditioning surfactant to fatty alcohol material is suitably from 10:1 to 1:10, preferably from 4:1 to 1:8, optimally from 1:1 to 1:7.

### OPTIONAL COMPONENTS

As optional components for inclusion in compositions according to the invention may be mentioned the following conventional adjunct materials known for use in cosmetic compositions: suspending agents, thickeners, pearlescing agents, opacifiers, salts, perfumes, buffering agents, colouring agents, emollients, moisturisers, foam stabilisers, sunscreen materials, antimicrobial agents, preservatives, antioxidants, natural oils and extracts, propellants. The invention will now be further illustrated by the following, non-limiting Examples.

### EXAMPLE

Two formulations were prepared having ingredients as shown in the following Table. Formulation 1 is a comparative example and Formulation 2 is an example according to the invention.

| Ingredient | Formulation 1 | Formulation 2 |
|---|---|---|
| Chemical Name | a.i. % | a.i. % |
| SLES 2EO | 14 | 14 |
| Cocoamidopropylbetaine | 2 | 2 |
| Guar | 0.1 | 0.1 |
| hydroxypropyltrimonium | | |
| chloride | | |
| Dimethiconol | 1 | 1 |
| Crosslinked polyacrylic | 0.4 | 0.4 |
| acid | | |
| Polytetrafluoroethylene | 0 | 2 |
| (PTFE) | | |
| Mica + titanium dioxide | 0.2 | 0.2 |
| Sodium benzoate | 0.5 | 0.5 |
| Water | to 100 | to 100 |

Comparative testing of formulations 1 and 2 showed that the presence of PTFE microparticles as in formulation 2 significantly increased dimethiconol (silicone) deposition onto hair, by around 60% compared to that observed for formulation 1. Deposition was measured by x-ray fluorescence spectroscopy. Furthermore, the friction coefficient of hair treated with formulation 2 was found to be significantly reduced compared with hair treated with formulation 1. Friction coefficients were measured using a friction rig developed for measurements on switches of hair fibres sliding against skin. Multiple measurements of frictional force were made on multiple switches treated with a particular formulation at each applied load. These data were used to produce representative coefficients of friction for each treatment.

What is claimed is:

1. An aqueous hair treatment composition comprising:
(i) at least one surfactant;
(ii) at least one dispersed insoluble benefit agent, and
(iii) PTFE microparticles.

2. A composition according to claim 1, in which said benefit agent is selected from the group consisting of conditioning agents, solid active agents, and mixtures thereof.

3. A composition according to claim 2, in which said conditioning agent is selected from the group consisting of silicones, high molecular weight hydrocarbon materials, oily or fatty materials, and mixtures thereof.

4. A composition according to claim 2, in which said solid active agent is an antimicrobial agent.

5. A composition according to claim 1, which is in the form of a shampoo composition comprising one or more anionic cleansing surfactants.

6. A composition according to claim 5, which further comprises a cationic polymer.

7. A composition according to claim 1, which is in the form of a hair conditioner comprising one or more cationic conditioning surfactants.

8. A composition according to claim 7, which further comprises a fatty alcohol material.

9. A composition according to claim 1, in which said PTFE microparticles are present in the form of a colloidal dispersion thereof.

10. A composition according to claim 9, in which the primary particle size of said PTFE microparticles ranges from 0.05 up to 0.5 microns.

11. Use of PTFE microparticles as a deposition aid for a dispersed insoluble benefit agent in an aqueous hair treatment composition.
